(19) United States
(12) Patent Application Publication
(10) Pub. No.: US 2022/0312902 A1
(43) Pub. Date: Oct. 6, 2022

(54) METHOD AND SYSTEM FOR CALCULATING PERSONALISED VALUES OF PARAMETERS OF A SOLE WITH A VIEW TO DESIGNING MADE-TO-MEASURE SOLES

(71) Applicant: ZHOR TECH, Nancy (FR)

(72) Inventor: Karim OUMNIA, Nancy (FR)

(21) Appl. No.: 17/763,286

(22) PCT Filed: Sep. 24, 2020
(86) PCT No.: PCT/EP2020/076821

§ 371 Date: Jun. 13, 2022

(30) Foreign Application Priority Data
Sep. 24, 2019 (EP) 19306195.9
Apr. 14, 2020 (EP) 20169506.1

(51) Int. Cl. A43D 119/00; A43D 1/02; B33Y 50/00; B33Y 30/00; B29C 64/386

(52) U.S. Cl. CPC A43D 119/00; A43D 1/02; B33Y 50/00; B33Y 30/00; B29C 64/386; B29L 2031/504

(57) ABSTRACT

The invention relates to a method (500) for calculating personalized parameter values of a new custom sole for the design of custom soles, said method comprising:
a step of loading (530) posture or mobility parameter values (101) of a user;
a step of loading (540) shoe parameter values (201);
a step of calculating (550) one or more personalized parameter values of a new custom sole (301).

The invention relates to the field of footwear items and their design, the invention can find an application in the monitoring of daily or sporting activities, or else the monitoring of the physiological state of the subject of study so as to determine personalized sole parameter values for the design of custom soles. The invention relates to a method for calculating personalized sole parameter values for the design of custom soles.

### PRIOR ART

The foot is a particularly complex part of the human body due to its composition since it has 26 bones, 107 ligaments and nearly 19 muscles. It also plays a particularly important role since it is the keystone allowing a human being to move. The slightest discomfort or the slightest degradation of the latter can quickly be disabling. This is particularly true when practicing a sport involving foot contact with the ground, which is found in a large part of land sports. In these sports, foot injuries can occur as a result of improper practice. However, injuries can also occur as a result of inappropriate use of footwear items, that is to say footwear items that do not have the technical characteristics (cushioning, flexibility, sole intended for a particular type of stride, etc.) adapted to the practice of sport by the person. Today, the consumer is faced with a variety of footwear choices and selections that were not available before. Although standard human foot morphologies exist, the fact remains that each foot is unique and may not correspond to existing standards. Thus the use of footwear items that are poorly or not adapted to a person's foot will tend to cause damage to the person's feet in the medium or long term. Historically, plantar orthoses have been developed to correct posture, biomechanical imbalance, legs of unequal length that can lead to scoliosis, or osteoarthritis pain in the knee. In addition to plantar orthotics, custom footwear items can also be made, usually by a health specialist. For this purpose, a well-known procedure for making footwear items consists in taking a measurement on the foot of the future wearer of the footwear item in order to determine all the morphometric parameters of the foot. In the event of incorrect positioning of the foot, a cast or impression of the foot is then made, according to which an orthopedic shoe insert is made. By means of the determined dimensions of the foot, a flat leather blank is cut to size. A dimensioned foot model is then made. The leather blank is then stretched over this model and the sole is glued to it, usually by sewing and/or gluing. This artisanal manufacturing process is particularly complicated and time-consuming to implement, in particular when a replica model of the foot is produced. Similarly, the production of an adjusted footwear item takes between 4 and 6 weeks and requires the intervention of health specialists. Thus, in order to provide a footwear item adapted to the foot of a user, a solution, described in patent application U.S. Ser. No. 10/148,700, has been developed. The solution thus described relates to a method for manufacturing adjusted footwear items, said method comprising the use of a prepared sole, a preformed rod and an insert. The pre-manufactured sole has dimensions corresponding to those of the foot intended to carry the footwear item. The insert is then applied to the pre-manufactured sole and the adjustment between the pre-manufactured sole and the insert applied to it is checked on the foot intended to wear the footwear item. The preformed rod is placed on the foot intended to wear the footwear item and is joined to the pre-manufactured sole so as to form a connection. However, this solution does not allow to take into account, in addition to the morphometric parameters of the wearer's feet, parameters associated with the gait of said wearer.

Another solution described in patent application CN105243547 aims at providing a service platform for personalizing footwear items. Such a platform thus comprises an information collection system, a data processing system and a manufacturing system. The information collection system is configured to collect user's gait data, three-dimensional foot data and shoe selection information, and send the user's gait data, three-dimensional foot data and shoe selection information to a data processing system. The information collection system mainly consists of a plurality of sensors placed within the sole of the footwear item. The data processing system is used to analyze and process the user's gait data, three-dimensional foot shape data and shoe selection information, and send the analysis and processing result to a manufacturing system. Regarding the shoe selection information, the user selects a brand online according to his own needs, he also selects the style and material of the shoe. However, this solution remains incomplete since it only takes into account the pressure exerted by the wearer of the footwear item, and does not offer a solution completely integrated into the sole of the footwear item.

The methods of the prior art are only based on incomplete analysis of the gait of a user of footwear items. Furthermore, the information collected is generally fragmented and does not allow to contextualize said information.

Thus, the existing solutions do not take into account the evolution over time of an individual's posture and mobility, particularly in relation to the soles he uses. There is therefore a need for new solutions allowing to offer personalized footwear items to a user, taking into account on the one hand the user's gait and on the other hand the footwear item parameters worn by said user.

### TECHNICAL PROBLEM

The purpose of the invention is therefore to overcome the disadvantages of the prior art. In particular, the purpose of the invention is to propose a method for calculating personalized sole parameter values for the design of custom soles, taking into consideration the posture or mobility parameters of the user and the footwear item parameters worn by said user.

### BRIEF DESCRIPTION OF THE INVENTION

The invention relates in particular to a method for calculating personalized parameter values of the custom soles for a user, said calculation method including the execution, by one or more computing devices, of the following steps:
a step of obtaining posture or mobility parameter values of the user, said posture or mobility parameter values having been calculated from raw data generated by at least one connected sole associated with a footwear item used by said user;
a step of obtaining plantar morphology parameter values of the user;
a step of obtaining user activity parameter values;
a step of obtaining new shoe parameter values, said new shoe parameter values including structural and/or geometric parameter values of a footwear item intended to receive the custom soles; and
a step of calculating one or more personalized parameter values of the custom soles for the user, said personalized parameter value(s) being calculated from the posture or mobility parameter values, the plantar morphology parameter values of the user, user activity parameter values and structural and/or geometric parameter values of the footwear item intended to receive the custom soles.

In particular, the invention relates to a method for calculating personalized parameter values of a new sole for the design of custom soles, said calculation method including the execution, by one or more computing devices, of the following steps:
a step of loading posture or mobility parameter values of a user, said posture or mobility parameter values having been calculated from raw data generated by at least one connected sole associated with a footwear item used by said user;
a step of loading shoe parameter values, said shoe parameter values including structural and/or geometric parameter values of the footwear item used by the user, associated with the connected sole;
a step of calculating one or more personalized parameter values of a new sole, said personalized parameter value(s) of the new sole being calculated from the posture or mobility parameter values and from the shoe parameter values;
a step of receiving and analyzing information from third-party users of third-party footwear items, said third-party user information including for each third-party user of third-party footwear items:
   third-party posture or mobility parameter values calculated from raw data generated by at least one third-party connected sole associated with a third-party footwear item, and
   third-party shoe parameter values of the third-party footwear item associated with the at least one third-party connected sole;
a step of identifying adjusted sole parameter values, said identification step including a comparison of the posture or mobility parameter values generated by the connected sole and the shoe parameters of the footwear item associated with said connected sole, to previously analyzed third-party user information.

Thus, a method according to the invention will be able to propose sole parameter values actually adapted to a user according to data directly generated during his movements but also according to data from other users. This allows to further improve the level of personalization and performance while benefiting from information sourced from other users who may have similar approaches.

According to other optional characteristics of a method according to the invention, said method may optionally include one or more of the following characteristics, alone or in combination:
it further includes a step of loading old shoe parameter values, said old shoe parameter values including structural and/or geometric parameter values of the footwear item used by the user associated with the connected sole (10) and said personalized parameter value(s) of the custom soles are further calculated from the old shoe parameter values. Thus, the characteristics of the shoes previously worn by the user are also taken into consideration when acquiring their postures, mobility and/or activities. As will be detailed later in the application, this can be combined with other qualitative data such as the evaluation by the user of the aspect of comfort, fatigue, pain, etc. Thus, the custom soles will be best suited to the user.
it further includes:
a step of receiving and analyzing information from third-party users of third-party footwear items, said third-party user information including for each third-party user of third-party footwear items:
   third-party posture or mobility parameter values calculated from raw data generated by at least one third-party connected sole associated with a third-party footwear item, and
   old third-party shoe parameter values of the third-party footwear item associated with the at least one third-party connected sole;
a step of identifying adjusted sole parameter values, said identification step including a comparison of the posture or mobility parameter values of the user, with information from third-party users analyzed beforehand.

In particular, this allows to improve values through the use of feedback from an entire user population. Advantageously, such data could possibly be labeled and processed through a learning model.
the third-party user information further includes a satisfaction index value of a third-party user, preferably with respect to the third-party connected sole and/or the footwear item including said third-party connected sole. The presence of such an index and then optionally its use during a step of identifying sole parameter values allows, for example, to select only the values associated with index values greater than a predetermined threshold or else to assign a weighting coefficient according to the value of the satisfaction index. Thus, the parameter values of the new custom sole are improved as well as the satisfaction of the user. For example, the step of identifying adjusted sole parameter values can be implemented only for third-party user information including a satisfaction index value and/or a correlation index respectively greater than a predetermined threshold.
the method comprises a step of determining a correlation index, for each third-party connected sole, between the third-party posture or mobility parameter values, the third-party shoe parameter values and the third-party user satisfaction index. Such a step improves the new sole parameter values as well as user satisfaction. Indeed, only the opinions of third-party users whose use is in line with the use recommended for a third-party connected sole equipping a footwear item can be taken into account. It is thus possible to highlight the connected soles or the footwear items having shoe parameters considered to be the most suitable and for proper use. Thus, the method can include a correlation between posture or mobility parameters with shoe parameters and satisfaction data. It will be possible to determine in the context of the present invention what is the preferred sole flexibility value of users having a median or average stride length greater than 1.5 m. Third-party shoe parameter values can include (structural and geometric) sole parameters.
The method includes the step of identifying adjusted sole parameter values is implemented only for third-party user information including a satisfaction index value and/or a correlation index respectively greater than a predetermined threshold. This advantageously allows not to take into account the parameter values originating from footwear items or from third-party soles that are potentially unsuitable for the user.
the method comprises a step, prior to the loading step, of processing raw data generated by the at least one connected sole used by the user, said processing step allowing the generation of posture or mobility parameter values. This processing step can be performed at the units, at a mobile device and/or at a server.
the posture or mobility parameters are selected from the impact force in contact with the ground, the pronation and/or supination and/or lameness parameters. These parameters when used allow the generation of particularly adapted parameter values of the new sole. This can be supplemented in particular by the main activity for which the custom sole is intended.
The method includes a step of generating a value of a first comfort indicator of a first sole, said first comfort indicator being generated following the entry of data by the user, via a man-machine interface of a presentation computing device and in that the step of calculating one or more personalized parameter values of the custom sole takes into account the generated value of the first comfort indicator. Such a step improves the new sole parameter values as well as user satisfaction. Furthermore, a comfort value indicated by the user can be correlated with biomechanical parameters (impact force, uppination, etc.) and more precisely with the variability of these parameters over a time window. Thus, it is possible for a system or a method according to the invention to implement time series techniques and/or control charts to follow the variability called natural variability (what is called common causes) versus the variability induced by events (special causes), which amounts to detecting patterns of discomfort in time series. These characteristics can preferably be applied to a pair of soles.
The method includes a step of generating a value of a second comfort indicator of a second sole, said second comfort indicator being generated following the entry of data by the user of said second sole, via the man-machine interface of the presentation computing device and in that the step of calculating one or more personalized parameter values of the custom sole takes into account the generated value of the second comfort indicator. Such a step improves the new sole parameter values as well as user satisfaction. These characteristics can preferably be applied to a pair of soles.
the step of calculating one or more personalized parameter values of the new sole, further comprises a comparison of the posture or mobility parameter values and the shoe parameter values previously loaded with the information of third-party users. Such a step improves the new sole parameter values as well as user satisfaction.
the step of identifying adjusted sole parameter values includes the use of a repository specifying target values or target ranges for the personalized parameter values of the new sole based on the posture or mobility parameter values generated and shoe parameter values acquired. Such a step improves user satisfaction.
it includes a step of calculating at least one use parameter value from the posture or mobility parameter values and in that the step of identifying adjusted sole parameter values takes into account the at least one calculated use parameter value. Such a step improves the new sole parameter values as well as user satisfaction.

The posture or mobility parameter values have been calculated by one or more processors integrated into the at least one connected sole. This allows to secure the data relating to the gait of users of connected soles and also to reduce the resource requirements for the systems for analyzing the use of footwear items.

The invention also relates to a method for manufacturing a new custom sole by a three-dimensional printing device, said method including the following steps:
Loading, by the printing device, a configuration file including one or more personalized parameter values of the new sole calculated during the implementation of a method for calculating personalized parameter values of a new sole according to the invention;
Generating, from the personalized parameter values of the new sole, a digital model of the custom sole;
Printing a custom sole from the generated digital model.

According to a third aspect, the invention relates to a system for calculating personalized parameter values of a new sole for the design of custom soles, said system comprising at least one calculation computing device and one footwear item comprising a connected sole, said at least one calculation computing device being configured to load posture or mobility parameter values calculated from raw data generated by at least one connected sole of a footwear item used by a user, load shoe parameter values, calculate one or more personalized parameter values of a new sole from the posture or mobility parameter values and the shoe parameter values, preferably receiving and analyzing information from third-party users of third-party footwear items equipped with third-party connected soles, and preferably identify adjusted sole parameter values.

Furthermore, a system for calculating personalized parameter values of a custom sole for the design of custom soles according to the invention may include at least one calculation computing device, said at least one calculation computing device being configured to obtain posture or mobility parameter values of the user, said posture or mobility parameter values having been calculated from raw data generated by at least one connected sole associated with a footwear item used by said user; Obtain plantar morphology parameter values of the user; Obtain user activity parameter values; Obtain new shoe parameter values, said new shoe parameter values including structural and/or geometric parameter values of a footwear item intended to receive the custom soles; and calculate one or more personalized parameter values of the custom soles for the user, said personalized parameter value(s) being calculated from the posture or mobility parameter values, the plantar morphology parameter values of the user, user activity parameter values and structural and/or geometric parameter values of the footwear item intended to receive the custom soles.

According to a fourth aspect, the invention relates to a three-dimensional printing system for the manufacture of new custom soles, said device including a processor capable of:
obtaining posture or mobility parameter values, plantar morphology parameter values of the user, user activity parameter values and structural and/or geometric parameter values of the footwear item designed to receive custom soles,
calculating one or more personalized parameter values of the custom soles for the user from the parameter values obtained,
generating a digital model of the custom sole;
   and a printing device configured to print the new custom soles from the generated digital model.

Preferably, the three-dimensional printing system according to the invention may include a scanner capable of generating parameter values of the plantar morphology of the user.

According to a fifth aspect, the invention relates to a computer program product comprising program instructions which, when executed by a processing unit of a computing device, cause the implementation of a method for calculating personalized parameter values of a new sole for the design of custom soles according to the invention.

Other advantages and characteristics of the invention will appear upon reading the following description given by way of illustrative and non-limiting example, with reference to the appended Figures:

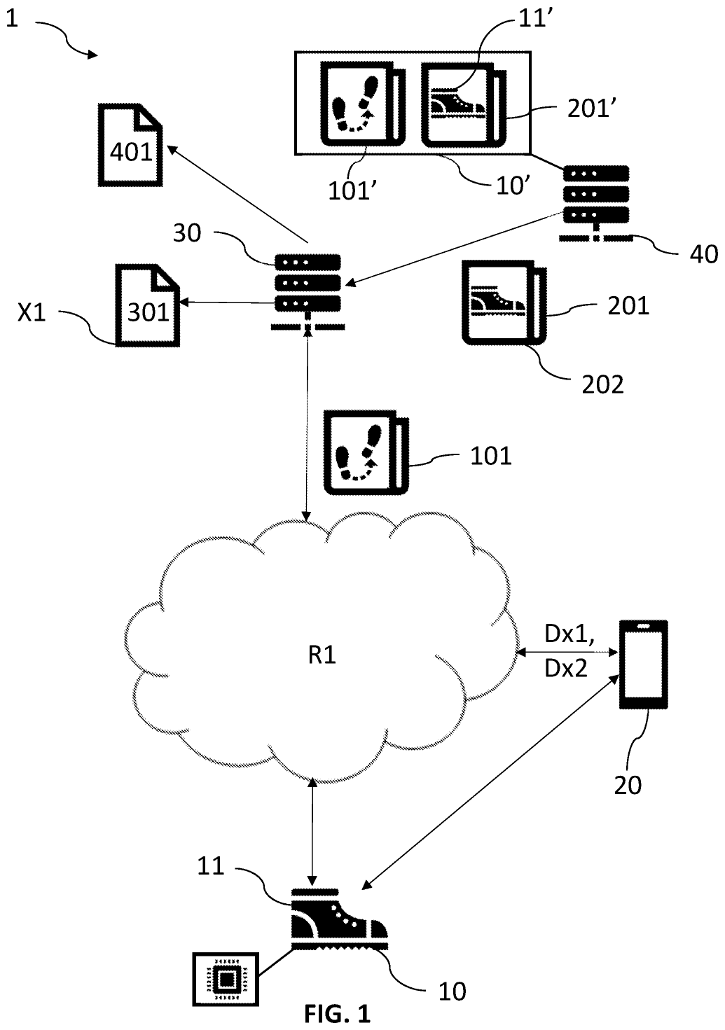

FIG. 1 shows an illustrative diagram of a system within which a method for calculating personalized sole parameter values for the design of custom soles in accordance with the invention can be implemented.

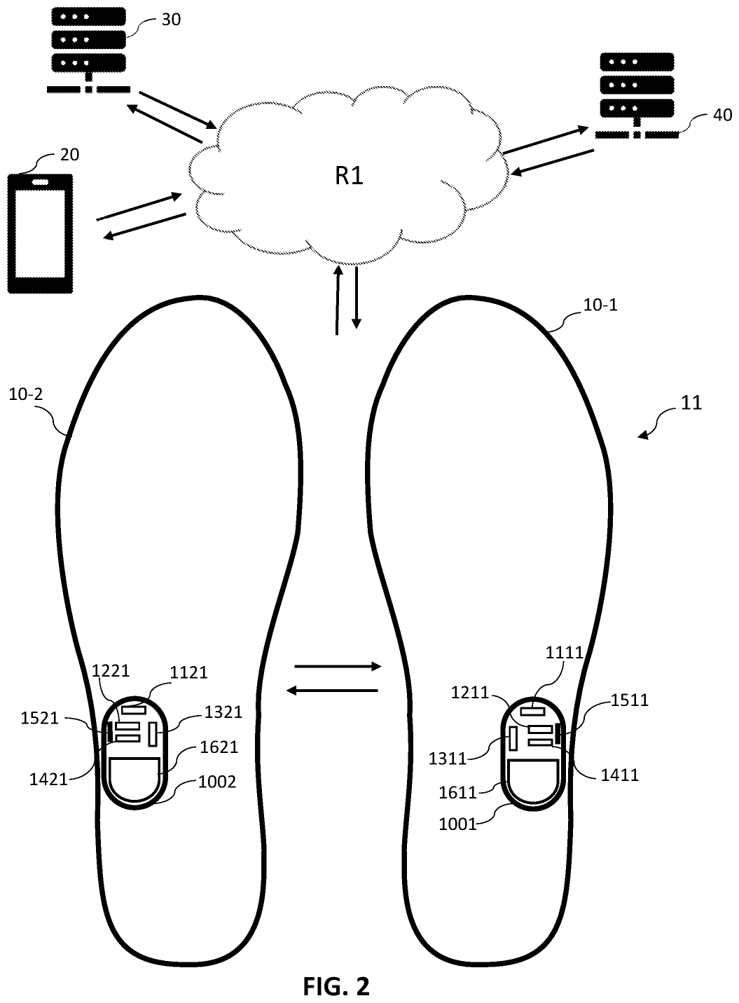

FIG. 2 shows a system for analyzing the use of footwear items used in the context of the invention and detailing an electronic unit of a connected sole.

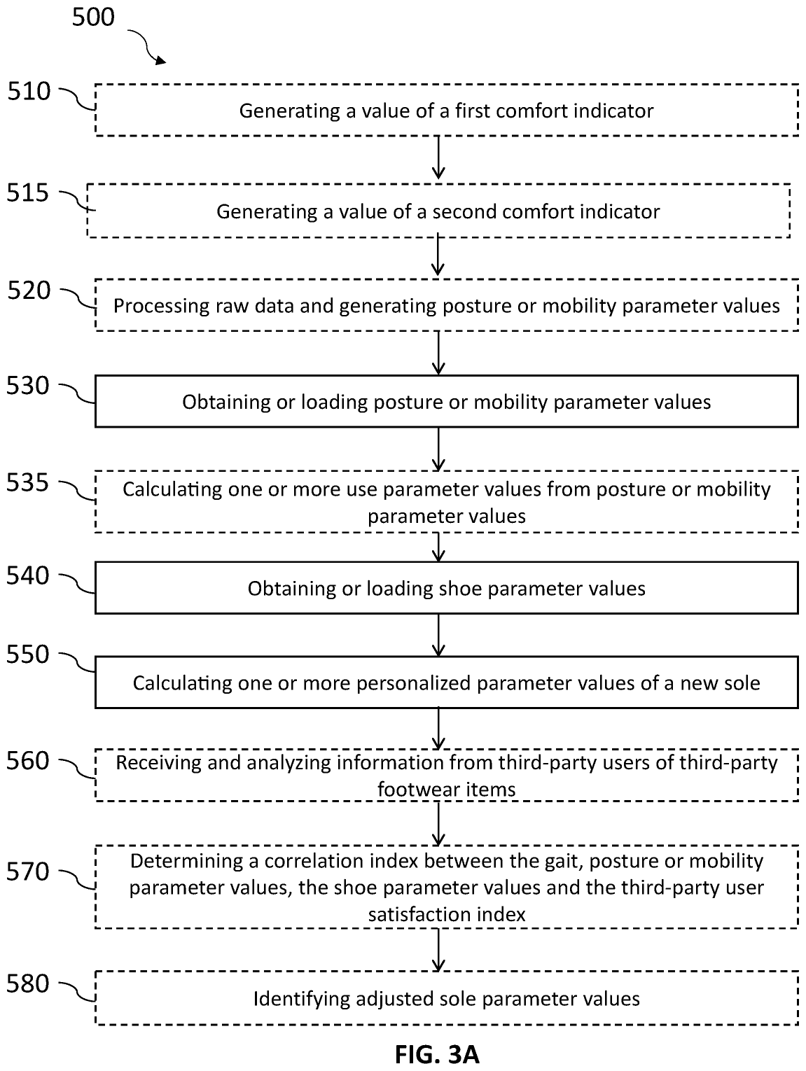

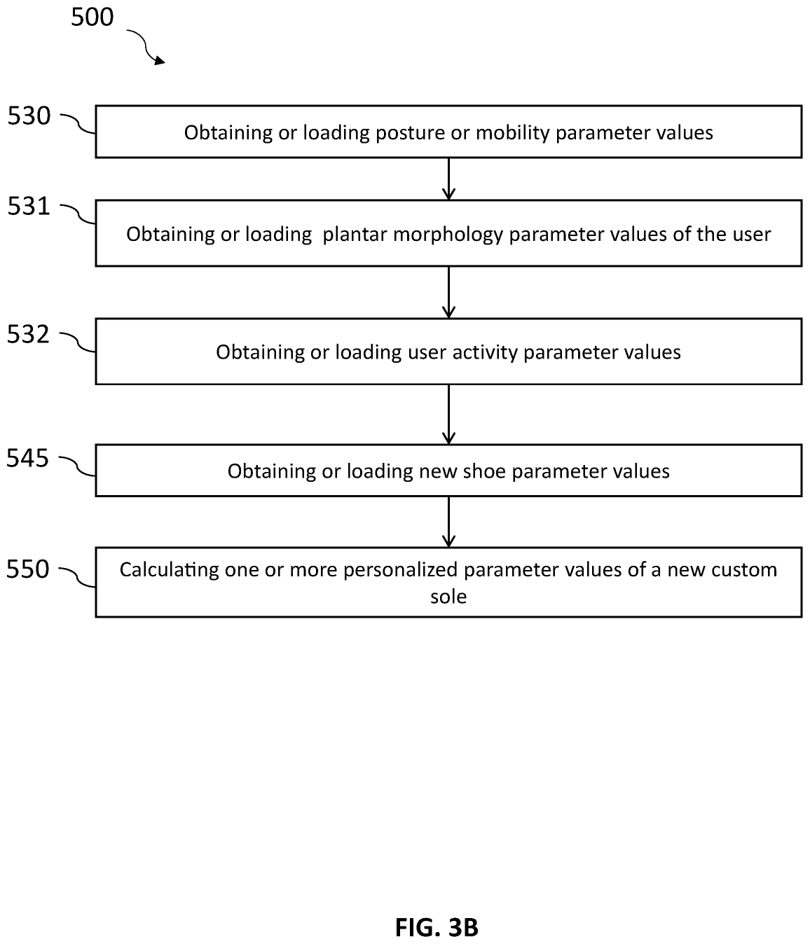

FIGS. 3A and 3B show illustrative diagrams of methods for calculating personalized sole parameter values for the design of custom soles in accordance with the invention.

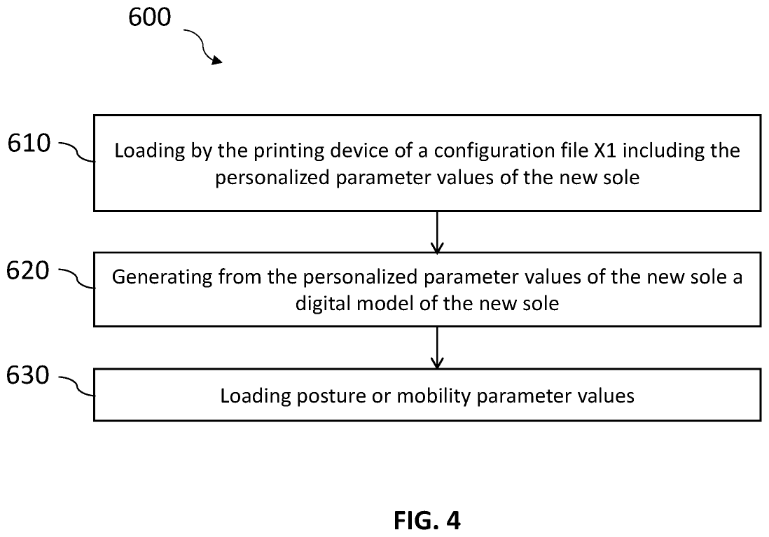

FIG. 4 shows an illustrative diagram of a method for manufacturing a custom sole in accordance with the invention by a three-dimensional printing device.

Aspects of the present invention are described with reference to flow charts and/or block diagrams of methods, apparatuses (systems), and computer program products according to embodiments of the invention.

In the figures, flowcharts and block diagrams illustrate the architecture, functionality, and operation of possible implementations of systems, methods, and computer program products according to various embodiments of the present invention. In this regard, each block in the flowcharts or block diagrams may represent a system, device, module, or code, which comprises one or more executable instructions to implement the specified logical function(s). In some implementations, the functions associated with the blocks may appear in a different order than that shown in the figures. For example, two blocks shown in succession may, in fact, be executed substantially simultaneously, or the blocks may sometimes be executed in reverse order, depending on the functionality involved. Each block in the block diagrams and/or flowchart, and combinations of blocks in the block diagrams and/or flowchart, may be implemented by special hardware systems that execute the specified functions or acts or perform combinations of special equipment and computer instructions.

### DESCRIPTION OF THE INVENTION

In the remainder of the description, “mobility” or “gait” within the meaning of the invention corresponds to the posture, movements, locomotion, and balance of the user. Balance corresponds in particular to postural balance related to the stability of the body and more particularly to the stability of a user's center of gravity. Nevertheless, it can integrate both static and dynamic balance.

The expression “posture or mobility parameters” corresponds to biomechanical parameters identified in the static or dynamic position.

The expression “movement analysis”, “mobility analysis” or “gait analysis” corresponds, within the meaning of the invention, to the attribution of one or more values, for example a score, a classification or a rating to a trajectory or to the displacement of a foot of a user. This characterization of the gait allows to obtain one or more numerical or alphanumeric values of biomechanical parameters representative of the gait.

The expression “raw data” corresponds to data generated by sensors and which has not yet been transformed. This may for example correspond to data generated by an inertial platform. The processing of raw data can allow to obtain biomechanical parameter values.

“Biomechanical parameter” means in the sense of the invention a characteristic of the posture or mobility of the user. A biomechanical parameter can be determined by various calculation operations from values of gait parameters generated by sensors of a connected sole. “Advanced biomechanical parameter” means in the sense of the invention a characteristic of the posture or mobility of the user determined at a key moment of a walking and/or running cycle and therefore more complex to be determined. A cycle being able for example to be a walking cycle. There are different types of activities such as pace, climbing a step, descending a step, stride, jump, flat, droop, stomp, kneel . . . . Therefore, a cycle can also correspond to a plurality of activities of different types depending on the complexity of the movement performed by the user.

“Sole” means an object allowing to separate the user's foot from the ground. A shoe can include an upper sole layer in direct contact with the foot of the user and a lower sole layer in direct contact with the ground or more generally the external environment. A shoe may also include a removable inner sole.

“Substantially identical” within the meaning of the invention means a value varying by less than 30% with respect to the compared value, preferably by less than 20%, even more preferably by less than 10%.

“Removable” means the ability to be easily detached, removed or disassembled without having to destroy the fastening means either because there is no fastening means or because the fastening means are easily and quickly removable (e.g. notch, screw, tab, lug, clips). For example, by removable, it should be understood that the object is not fixed by welding or by any other means not intended to allow the object to be detached.

“Process”, “calculate”, “determine”, “display”, “transform”, “extract”, “compare” or more broadly “executable operation” mean, within the meaning of the invention, an action performed by a device or processor unless the context indicates otherwise. In this regard, operations refer to actions and/or processes of a data processing system, for example a computing system or an electronic computing device, which manipulates and transforms data represented as physical (electronic) quantities in computing system memories or other information storage, transmission or display devices. These operations can be based on applications or software.

The term “learning” within the meaning of the invention corresponds to a method designed to define one or more correspondences, which may or may not take the form of a function f, allowing to calculate a value of Y from a base of n labeled (X1 . . . n, Y1 . . . n) or unlabeled (X1 . . . n) observations. Such a correspondence or function may correspond to a prediction model. Learning can be said to be supervised when it is based on labeled observations and unsupervised when it is based on unlabeled observations. In the context of the present invention, learning is advantageously used for calculating one or more personalized parameter values of a new custom sole.

“Prediction model” means any mathematical model that allows to analyze a volume of data and to establish relationships between factors allowing the assessment of risks or that of opportunities associated with a specific set of conditions, in order to guide decision-making towards a specific action.

The terms or expressions “application”, “software”, “program code”, and “executable code” mean any expression, code or notation of a set of instructions intended to cause data processing to perform a particular function directly or indirectly (e.g. after a conversion operation to another code). The examples of program code may include, but is not limited to, a subroutine, function, an executable application, a source code, an object code, a library, and/or any other sequence of instructions designed for the running on a computing system.

Within the meaning of the invention, the term “processor” designates at least one hardware circuit configured to execute instructions contained in the program code. The hardware circuit may be an integrated circuit. Examples of a processor comprise, but are not limited to, a central processing unit (CPU), a network processor, a vector processor, a digital signal processor (DSP), a field programmable grid network (FPGA), a programmable logic assembly (PLA), an application specific integrated circuit (ASIC), a programmable logic circuit and a controller.

“Coupled”, within the meaning of the invention, means connected, directly or indirectly with one or more intermediate elements. Two elements can be coupled mechanically, electrically or connected by a communication channel.

In the remainder of the description, the same references are used to designate the same elements. Furthermore, the different characteristics presented and/or claimed can be advantageously combined. Their presence in the description or in different dependent claims does not exclude this possibility.

As already mentioned previously, the invention allows to personalize the soles on the basis of the walking biomechanics of a user, which could also be combined with the composition, the material forming the soles used by the user, in order to offer soles with a shape perfectly adapted to the user but also with combinations of materials of densities and other rebounds adapted to the movements of the user.

Although many solutions have been developed to allow the personalization of soles for each user, in particular with a view to offering products and/or services as close as possible to their needs, the result is that, very often, the footwear item is not necessarily ideally arranged to receive the personalized sole. Indeed, the parameters related to the footwear item(s) intended to receive a personalized sole are generally not taken into account for the design of the personalized sole. However, in general, and more particularly in the field of sports involving walking or running, a user or more commonly an athlete generally seeks to equip himself with the most suitable footwear items for the way in which he practices a sport. This is particularly true for footwear items for which an unsuitable choice can have consequences on the well-being of the user and more particularly on the performance delivered by the athlete who is equipped therewith. This can even cause heavy damage to the feet of said athlete. Indeed, each person has a very particular way of moving and it is recognized that there are mainly three main families of different strides: The universal stride, The pronator or over-pronator stride, The supinator or under-pronator stride.

Very often, to be able to identify a supinator or over-pronator stride, tests and analyzes of the stride by a specialist are necessary. Thus, many athletes do not know what type of stride they practice and whether it is pronounced or not. Added to this, the growing need for athletes or not, to equip their footwear item with orthopedic soles in order to rectify or avoid the appearance of a defect in their gait. As a result, they can easily choose footwear items that are poorly or not suited to a personalized sole, which can cause damage to their feet in the long term.

To address this problem, the applicant has developed a solution for calculating personalized parameter values for a new sole. Thus, it allows the design of custom soles automatically, in particular based on posture or mobility parameters of the future user of the new sole and the shoe parameters of the user. Furthermore, the solution can take into account posture or mobility parameters of third-party users with their shoe parameters in order to best adjust the personalized parameter values of the new sole.

Thus, according to a first aspect, the invention relates to a method 500 for calculating personalized parameter values of a new sole for the design of custom soles 301, said method being implemented within a computer system configured appropriately.

As mentioned, the custom sole 301 is advantageously produced on the basis of calculations of personalized values made from parameters obtained by means of the system illustrated by FIG. 1. The system uses for this purpose one or two connected soles.

As illustrated in FIG. 1, a system 1 for calculating personalized parameter values of a new sole for the design of custom soles comprises at least one calculation computing device 30 and a footwear item 11 comprising a connected sole 10.

Furthermore, a system 1 for calculating personalized parameter values of a new sole for the design of custom soles can also include a presentation computing device 20 and a third-party computing device 40.

Thus, in the context of the invention, a system 1 comprises a calculation computing device 30 configured to load posture or mobility parameter values 101 calculated from raw data generated by at least one connected sole 10 of a footwear item 11 used by a user, load shoe parameter values, calculate one or more personalized parameter values of a new sole 301 from the posture or mobility parameter values 101 and from the shoe parameter values. The loading of the parameter values can in particular correspond to the loading into memory of these data. A calculation computing device 30 may be configured to obtain these values and in certain cases it may be configured to calculate them for example from raw data.

The shoe parameter values used in the context of this calculation of one or more personalized parameter values of a new sole 301 may correspond to the shoe parameter values 201 used by the user associated with the connected sole 10 or with the new shoe parameter values 202. This calculation of one or more personalized parameter values of a new sole 301 can also include the use of plantar morphology parameter values of the user and user activity parameter values.

Furthermore, it can be configured to receive and analyze information from third-party users of third-party footwear items equipped with third-party connected soles 10′, and identify adjusted sole parameter values.

A calculation computing device 30 advantageously comprises a processing unit or a processor, for example in the form of a microcontroller cooperating with a data memory, possibly a program memory, said memories possibly being dissociated. Such a data memory can be configured to store a computer program whose program instructions, interpretable and executable by the processing unit, allow to automatically adapt a conventional computing device so that it becomes a calculation computing device 30 in accordance with the invention.

The data memory can be partially or entirely electrically erasable in order to be updated. Generally, a section of said data memory is not erasable by construction, or is protected against such erasure by a security mechanism. Such a memory section records in a durable manner, in particular the value of a unique identification datum characterizing a connected sole 10 with regard to other connected soles. The processing unit cooperates with said memories by means of an internal communication bus. Thus, the data from sensors positioned in said connected sole can be stored in such a data memory. Said data memory can further store personal data associated with the user of the connected sole. The calculation computing device 30 may be located in a cloud. Thus, the data from the sensor(s) positioned in the connected sole 10 can be related to the personal data of the user, through the value of the unique identification datum of the connected sole 10. The personal data associated with the user of the connected sole 10 can correspond to data accessible on request from a computing device configured to store such personal data. Advantageously, the personal data are entered by the user of the connected sole 10 via a dedicated application installed on a presentation computing device 20. Thus, the user can enter personal data such as his sex, his age, his weight, his height, his shoe size or more generally any morphometric or non-morphometric datum of interest in the context of the calculation of the values of the user's posture or mobility parameters. Thus, it is expected that the user can indicate, in the context of entering his personal information, one or more pathologies having an influence on his gait, or more generally any physical failure involving difficulty in moving. Such a pathology or physical failure can be selected via a list through the dedicated application or can be entered in a dedicated field. Such a pathology or such a physical failure may consist advantageously but in a non-limiting manner of articular problems of one or more limbs of the user, a hallux valgus, a hallux rigidus, a claw toe (“hammer toe”), a bunionette, Morton's syndrome, 2nd ray pain syndrome, intermetatarsal bursitis, sesamoidopathies, tendinopathies or any physical injury affecting the user's gait.

Such a calculation computing device 30 also comprises communication means configured to communicate through a long-range communication network R1 of the Internet, LoRa or Sigfox type or any other equivalent communication network. Advantageously, a calculation computing device 30 can correspond to a computing server or else to an electronic unit of a connected sole 10. Of course, a calculation computing device 30 cannot be limited to a computing server and may also correspond to a computer-type computing machine further comprising a man-machine interface, that is to say any element allowing a human being to communicate with a particular computer and without this list being exhaustive, a keyboard and means allowing, in response to commands entered on the keyboard, to perform displays and optionally to select, using the mouse or a touchpad, elements displayed on a screen. Another exemplary embodiment is a touch screen allowing to select directly on the screen the elements touched by the finger or an object and optionally with the possibility of displaying a virtual keyboard.

A calculation computing device 30 can advantageously communicate with other computing devices, such as a presentation computing device 20 or else with a connected sole 10 equipping a footwear item 11.

For this purpose, a footwear item 11 corresponds to a shoe intended to be worn by a user. Generally, the user will wear two footwear items 11, one on each foot. The footwear items 11 will be equipped with one, preferably two connected soles 10 (that is to say one connected sole 10 per footwear item).

Indeed, one of the advantages of the invention is to be able to rely on raw data generated by one or more connected soles 10 in order to be able to identify sole parameter values adjusted to the footwear item 11.

A connected sole 10 is configured to generate raw data from which it is possible to calculate posture or mobility parameter values 101. Such raw data can be sent directly to the calculation computing device 30 which will then be configured to calculate the posture or mobility parameter values 101 from the raw data received and to store them in its data memory. Provision is also made for the raw data generated by a connected sole 10 to be transmitted to a third-party computing device 40 which will then be configured to calculate the posture or mobility parameter values 101 from the raw data received. The posture or mobility parameter values 101 can thus be loaded either directly from the data memory of the calculation computing device 30 or from the third-party computing device 40. In a particular embodiment, a connected sole 10 may comprise hardware and software resources configured to calculate posture or mobility parameter values 101.

The posture or mobility parameter values 101 calculated from raw data from a connected sole 10 are generally generated in connection with identified biomechanical parameters of the user in a position called dynamic position, that is to say that the user performs at least one movement. In addition, the identified biomechanical parameters of the user can be associated with a position called static position, that is to say the user does not perform any movement. Such biomechanical parameters associated with a static position may in particular correspond to the personal information of the user entered in connection with morphometric data of the foot (shape, dimensions, size, etc.) of the user. The posture or mobility parameter values associated with a static position will thus be able to be compared with the posture or mobility parameter values associated with a dynamic position in order to demonstrate whether or not said values match. Indeed, depending on the posture or mobility parameter values associated with a static position, in particular when the latter reveal a pathology, it can be expected that the posture or mobility parameter values associated with a dynamic position reveal a gait disorder associated with said pathology.

Furthermore, posture or mobility parameter values can be determined from specific exercises performed by the user. Such exercises are for example walking or stair climbing steps. Thus, a dynamic type posture or mobility parameter value can represent a movement of a user such as, by way of non-limiting example, a “pace” and a static type posture or mobility parameter value can, advantageously but in a non-limiting manner, represent a posture of the “kneeling” type of a user. There are different types of exercises such as the pace, climbing a step, descending a step, stride, jump, flat, droop, stomp, kneel . . . . Therefore, it is possible to determine a plurality of posture or mobility parameter values from such exercises such as in particular the movement of the foot in the footwear item and thus measure the rotation of the ankle of the user and especially the level of support that offers said footwear item. The person could also be asked to do other exercises to identify more information about flexibility, cushioning etc. of the footwear item.

The posture or mobility parameters 101 can correspond to biomechanical parameters. Thus, the posture or mobility parameters 101 can be selected, for example, from: pronation/supination values, impact force values, pace length values, contact time values, acceleration, angular speed values, sole orientation values, propulsion speed, fatigue rate, Fick angle, a propulsion direction and a deceleration direction. Such parameters may also correspond to the pace length, the contact time, time of flight, lameness, propulsion force, balance and several other parameters relating to the user and describing his gait, postures and his movements.

Advantageously, the most relevant posture or mobility parameters 101 in the context of the present invention are: the parameters related to the health of the person such as the impact force parameter in contact with the ground (in particular if it shows a high value), pronation and/or supination parameters (in particular if they show a high value), and/or lameness. Furthermore, the shoe wear parameter is also very relevant, in particular because it can create injuries.

Even more preferably, the posture or mobility parameters 101 include at least: the impact force parameter the pronation parameter and/or the supination parameter.

As illustrated in FIG. 2, it will be assumed that a footwear item 11 comprises a pair of shoes equipped with a pair of connected soles. In each of said shoes, an electronic unit 1001, 1002 is arranged in a sole of each of said shoes, thus corresponding to a connected sole referenced 10-1, 10-2 in connection with FIG. 2.

The connected soles can for example correspond to outer soles or to inner soles of a footwear item. These soles can be removable or be permanently integrated into the sole assembly of the footwear item.

Conventionally, the connected soles 10-1, 10-2 each include an electronic unit 1001,1002. As shown in FIG. 2, the electronic unit 1001,1002 is preferably positioned at a middle portion of the sole.

An electronic unit 1001, 1002 advantageously weighs only a few grams and has a reduced size adapted for a sole of a shoe, this electronic unit 1001, 1002 is housed in a space-saving manner in any inner and/or outer sole. This low volume limits the impact on user comfort and has the advantage of optimizing production costs by making it less expensive and simpler to integrate this technology into the sole during the industrial process.

The choice of material for the electronic unit 1001, 1002 is made in such a way as to ensure its solidity as well as the possibility of inserting it into a sole. Indeed, it is necessary to be able to manufacture a product which can, on the one hand, withstand the weight of a person and, on the other hand, be easily inserted into a sole or a shoe. Combining miniaturization and resistance of the unit is a real challenge: many prototypes had to be made before determining the material that would allow such a unit to be inserted into a sole, without altering the comfort of the latter.

Such an electronic unit 1001, 1002 includes an inertial platform 1111, 1121 configured to generate a set of data (e.g raw data) on the gait of a user wearing a footwear item 11 including at least one connected sole 10. In particular, the inertial platform 1111, 1121 is configured to generate a set of data on a movement of the foot of a user equipped with the connected sole 10.

While a user is walking, the inertial platform 1111, 1121 acquires signals representative of a movement parameter (acceleration and/or speed, for example angular speed) of the foot along the axes X, Y, Z. Furthermore, these data can then be processed to generate at least one acceleration signal.

The electronic unit 1001, 1002 can also include one or more magnetometers so as to acquire three additional raw signals corresponding to the magnetic field values in three dimensions.

Each electronic unit 1001, 1002 can also include other sensors, in particular an inclinometer, a barometer, a temperature sensor, a humidity sensor and an altimeter to benefit from increased precision. Furthermore, the electronic unit can be coupled to other sensors, for example distributed in the sole, such as pressure sensors or force sensors. In particular, the pressure and/or force sensors can include electrodes and be made of piezoelectric materials.

The inertial platform is for example made up of at least one accelerometer and one gyroscope. Preferably, it includes several accelerometers and gyroscopes. More preferably, the inertial platform 1111, 1121 includes at least one accelerometer and at least one gyroscope, and can be supplemented by other sensors, in particular a magnetometer, a barometer and an altimeter.

Furthermore, advantageously, the data generated by the electronic units 1001, 1002 are encrypted. In this case, advantageously, only the computing device intended to receive said generated data is configured to decrypt them.

In particular, the data generated by the electronic units 1001, 1002 are encrypted using public keys each associated with one of the electronic units and the calculation computing device 30 can have the private keys necessary for decrypting the data generated.

Furthermore, the electronic unit 1001, 1002 may include a data processing module 1211,1221 configured to transform all the data generated using predefined algorithms. Thus, the electronic units 1001, 1002 can be configured to process the signals generated by the inertial platform so as to facilitate subsequent processing by a computing device. The data received via the sensors located in the inner and/or outer soles are processed according to one or more algorithms in each of the electronic units. The processing module is advantageously configured to carry out a pre-processing of the data generated and optionally to carry out a sufficient processing to generate information on the posture or the gait of the user, information that the electronic unit transmits to a computing device, in real time or offline with the raw data.

The electronic units 1001, 1002 can also be configured as a slave unit, which receives the data generated by the sensors located in its sole/shoe and transmits them to the master unit, (also called the main unit), which receives the data from the slave unit, processes them by comparing them with its own data and generates information on the posture of the user in general and of his feet in particular, information that the master unit transmits to a computing device in real time or offline.

The processing module 1211,1221 allows to analyze in 3D the posture, the movements, the locomotion, the balance and the environment of the user, and more generally all that will be qualified as being his walk, from the data collected by the inertial platform and any additional sensors placed in the sole.

The processing module 1211, 1221 can be used to generate biomechanical gait parameters. Advantageously, the processing module 1211, 1221 is configured to transform the set of data into at least one posture or mobility parameter 101 such as those mentioned above.

Furthermore, the transformation by the processing module 1211, 1221 can advantageously comprise the segmentation of the data into a plurality of phases. Preferably, the data processing module 1211, 1221 is able to segment a pace into at least four phases such as: the impact phase (corresponds to the precise moment of contact of the foot with the ground), the bearing phase (takes place from the impact phase until the heel lifts off the ground), the propulsion phase (begins when the heel leaves the ground and ends when the first toe leaves the ground) and the flight phase (starts when the first toe leaves the ground and ends when the heel touches the ground).

More specifically, the division or segmentation of the pace can allow to identify the main bearing areas for the user. Thus, it is possible to measure the shape of the pace during walking or any other activity of the user in order to determine the possible malformations of the feet and postures of the user.

The information generated will then be transmitted to the second electronic unit 1002, or more generally to the master unit (main unit) by emitting signals which may, by way of non-limiting example, be of the Bluetooth type.

The electronic unit 1001, 1002 can advantageously be formed by encapsulating its components. For example, the encapsulation can be in the form of a conformal coating or a resin (e.g. silicone, epoxy, polyurethane). The encapsulation of all the components (e.g. inertial platform, processing module, . . . ) provides good insulation and thus combines good electrical properties with excellent mechanical protection.

When an electronic unit is not able to communicate in real time with the other unit and/or with the computing device, it stores the information collected and transmits it offline when the exchange is again possible. This offline transmission of the data collected is made possible thanks to the storage capacity with which each of the electronic units is equipped.

Thus, the electronic unit includes a data storage module 1311,1321, configured to memorize at least part of the transformed data and/or the data generated by the processing module. Such a data storage module 1311,1312 can advantageously have a memory capacity of less than 512 kB, preferably less than 128 kB, more preferably less than 32 kB and even more preferably less than 16 kB. In particular, the storage module can correspond to the memory available on a CPU.

Furthermore, the electronic unit 1001, 1002 includes first communication means. Thus, in particular, each of the electronic units is designed so as to be able to communicate independently with the other and/or directly with a computing device or else with a communicating electronic device in order to be able to exchange its own information on the posture/the movement/the activity of its foot, from which it received the data via the various sensors of the inner and/or outer sole of the shoe thus equipped.

Preferably, the electronic unit 1001, 1002 includes first communication means 1411, 1421 configured so that the electronic unit 1001, 1002 of at least one of the soles is able to transmit at least part of the raw data, in real time or offline, to a calculation computing device 30 or to a third-party computing device 40 or else to a presentation computing device 20. As presented, these data are preferably data called raw data, that is to say data as generated by the inertial platform (preferably 9 axes and at least at 200 Hz), but can also be preprocessed data or posture or mobility parameter values 101.

Advantageously, each electronic unit 1001, 1002 includes second communication means configured so that the electronic unit 1001 of a connected sole is able to communicate with the electronic unit 1002 of a second connected sole. Thus the electronic units 1001, 1002 will be able to exchange information in real time. Indeed, the generation of data by the inertial platforms must preferably be synchronized and this advantageously involves communication between the two electronic units 1001, 1002. More preferably, the electronic units 1001, 1002 are configured so as to be able to punctually check their synchronization.

In particular, the two electronic units 1001, 1002 are configured to communicate with each other and to initiate the generation of data on the movement of a user's foot only after receiving a message from the other electronic unit.

The first and second communication means are capable of receiving and transmitting the data over at least one communication network R1. Preferably the communication is operated via a wireless protocol such as wifi, 3G, 4G, 5G and/or Bluetooth. Preferably the communication protocol is a BLE or ANT+ protocol. These communication protocols allow low energy consumption.

Advantageously, each of the units 1001, 1002 is designed so as to be able to communicate with the second unit, for example by short wave or high frequency signals of the Bluetooth Low Energy or ANT+ type.

Thus, in particular, each of the units, whether slave or Master unit, is designed so as to be able to communicate independently with the other and/or directly with a computing device in order to be able to exchange its own information on the posture/the movement/the activity of its foot, from which it has received data via the various sensors of his inner and/or outer connected sole of a footwear item.

Advantageously, due to its confinement inside a unit placed under the body of a person, the antenna 1511, 1521 should preferably be disposed inside the unit on the side facing the outside of the sole. This positioning of the antenna is preferable insofar as laboratory tests have established that the signal emitted from a sole or shoe is 70% absorbed by the human body. This antenna must therefore be positioned on the periphery of the foot and oriented in such a way as to always be able to transmit the signal to the unit of the second sole. Preferably, the antenna can be an antenna printed on an electronic card. Alternatively, the antenna can be printed on an inner face of the unit and connected to the electronic card by wiring. The antenna can preferably be positioned on a low part with respect to the electronic card. Thus, the electronic card makes contact with the antenna.

Furthermore, the electronic unit 1001, 1002 includes an energy source 1611,1621. The energy source is preferably of the battery type, which is rechargeable or not. Preferably the power source is a rechargeable battery. Furthermore, it can be associated with a system for charging by movement or by external energy. The system for charging by external energy can in particular be a system for charging by wired connection, a system for charging by induction or else a photovoltaic system.

The electronic unit 1001, 1002 can include a power source 1611,1621 of the rechargeable battery type, the recharging of which can be carried out using different technologies:
by charger, with a connector flush with the sole;
with a mechanical recharging device integrated into the sole, such as for example a piezoelectric device capable of supplying electrical energy from walking;
with a contactless device, for example by induction; or
with a photovoltaic device.

Furthermore, the electronic unit according to the invention may include a wired connection means, preferably protected by a removable tab. Such a tab can preferably be made of a polymer of the elastomer or polyurethane type. This wired connection means can for example be a USB or firewire port. Advantageously, the USB port is also resistant to water or humidity.

This wired connection means can be used as mentioned above to recharge the battery but also to exchange data and for example update the firmware of the electronic card carrying the various components of the electronic unit.

These various components of the electronic unit are preferably arranged on an electronic card (or printed circuit). Furthermore, the various means and modules of the electronic unit 1001, 1002 are represented separately in FIG. 2, but the invention may provide for various types of arrangement such as for example a single module combining all the functions described here. Likewise, these means can be divided into several electronic cards or even combined on a single electronic card.

Furthermore, a system 1 used in the context of the invention comprises a presentation computing device 20 that can be configured to receive raw or preprocessed data, generated by a connected sole 10 or more particularly by an electronic unit 1001, 1002. The presentation computing device 20 is generally a tablet, a smartphone. It can be configured to transfer these data to a remote calculation computing device. It is then possible, for example, to access this remote computing device via a web interface.

Advantageously, a dedicated application is installed on the presentation computing device 20 in order to process the information transmitted by the units and allow the user to interact with the computing device in charge of processing the raw data generated by the connected sole 10. In particular, the user will be able to consult all the parameter values generated by the connected sole or else by the calculation computing device 30. Thus, a connected sole 10 can be associated, preferably coupled directly or indirectly, with a presentation computing device 20.

Thus, a method according to the invention includes the execution, by one or more computing devices 20, 30, 40, of a plurality of steps such as one or more of the steps described below. In the remainder of the description, the steps of a method in accordance with the invention, in particular described in connection with FIGS. 3A and 3B, are preferably implemented by a calculation computing device 30.

More particularly, a method in accordance with the invention comprises a step of obtaining or loading 530 posture or mobility parameter values 101 of a user, said posture or mobility parameter values having been calculated from raw data generated by at least one connected sole 10 associated with a footwear item 11 used by said user.

Advantageously, such posture or mobility parameter values are stored in memory in the data memory of the calculation computing device 30.

In a particular embodiment, however, provision is made for the posture or mobility parameter values 101 to be loaded directly from a third-party computing device 40 whose main function would be to generate the posture or mobility parameter values 101 to from the raw data generated and sent by the connected sole 10.

In some cases, it may be desirable to use certain posture or mobility parameters and not others, for example in the context of a user with a pathology affecting his gait, or else in the context of an athlete practicing a particular activity. A method in accordance with the invention may include a step 520, prior to the loading step 530, of processing raw data generated by the at least one connected sole 10 used by the user. This processing step 520 advantageously allows the generation of posture or mobility parameter values 101. Whether the raw data comes from a third-party computing device 40 or whether it is in the data memory of the calculation computing device 30, the calculation computing device 30 can be configured, from the raw data, to generate the posture or mobility parameter values 101 desired and most suitable for the user.

Furthermore, as illustrated in FIG. 3B, a method according to the invention may include a step of obtaining 531 or of loading plantar morphology parameter values of the user. The plantar morphology parameter values of users can be obtained from dedicated servers or from information presentation devices coupled to the connected sole. The plantar morphology parameters may preferably correspond to one or more photographs of the foot or else to a three-dimensional model of the foot. These plantar morphology parameters may include dimensions of the foot (length/height of the arch, length/width of the foot), size and position of the toes, and/or shape of the arch of the foot (e.g. sunken or exaggerated for respectively flat foot or hollow foot). Using plantar morphology parameter values of the user will improve user comfort when using custom soles.

Furthermore, as illustrated in FIG. 3B, a method according to the invention may include a step of obtaining 532 or loading user activity parameter values. The activity parameters may correspond in particular to the intended uses for the custom soles. For example, to types of activities, activity frequencies or activity intensities. It will be possible, for example, that a custom sole is intended to be associated with a shoe intended for a walking activity, with a high weekly frequency and with certain values of impact force and/or propulsion force.

Furthermore, the user activity parameters may correspond to a sporting activity of the user with this type of shoe (walking, running, basketball, . . . ).

In order to be able to determine which parameters must be taken into account for the manufacture of a new personalized sole, it may be advantageous to be able to determine directly from the posture or mobility parameters 101, for which use said new sole is intended. For this purpose, a method in accordance with the invention may include a step of calculating 535 at least one use parameter value 401 from the posture or mobility parameter values 101 generated.

Thus, the calculation computing device 30 can include, in a dedicated data memory, a use repository. The use repository includes reference posture or mobility values with which a predetermined type of use is associated. By way of non-limiting examples, the determination of a use, by the step of calculating 535 at least one use parameter value 401, can be associated with a pattern of posture or mobility values 101 of specific reference. Such determination of a use may also be subject to whether or not a predetermined threshold is exceeded. The person skilled in the art will appreciate that the possibilities of determining a use according to the posture or mobility parameters 101 are very numerous and that it will be possible to configure the calculation computing device 30 so that during the implementation of the calculation step 535, one or more use parameter values 401 are determined, depending on the posture or mobility parameters 101 deemed relevant for a given use. By way of non-limiting example, a use parameter value may indicate a sporting, urban, recreational use of a footwear item 11. For this purpose, the computer calculation device 30 can be configured to identify in the posture or mobility parameter values 101 previously loaded, values describing an impact force, a pace length, an acceleration, a propulsion speed and a time of flight. In particular, the calculation computing device 30 can be configured to compare, during the implementation of the calculation step 535, the values describing an impact force, a pace length, an acceleration, a propulsion speed and a time of flight. In particular, the calculation computing device 30 may be configured to compare values describing an impact force, a pace length, an acceleration, a propulsion speed, and a time of flight to a plurality of reference value patterns for these posture and mobility parameters taken alone or in combination. The person skilled in the art will understand that in the context of sports practice one or more parameters may be taken into account to identify a use of a footwear item. In particular, a sports practice of the running type could, for example, be characterized by values describing an acceleration, a particular time of flight or else a pace length or a running speed or else a combination of these parameters. In the context of problems more related to the field of health, affecting for example the gait of a user, other posture or mobility parameters may be taken into account to identify a given use. In particular, the gait or mobility parameters, affecting the user's gait, which can be analyzed are the presence of lameness, a fatigue rate, orientation values of the sole, balance. Thus, it will be possible to determine a particular use of a footwear item in the context of a sports practice for example.

In order for the new sole to be adapted to the footwear item 11 of the user, a method 500 in accordance with the invention may comprise a step of loading 540 shoe parameter values. The shoe parameter values may include structural and/or geometric parameter values of the footwear item 11. Furthermore, these shoe parameter values may correspond to shoe parameter values 201 used by the user associated with the connected sole 10 or else to new shoe parameter values 202 intended to receive the custom sole. The first shoe parameter values 201 used by the user may be useful for interpreting the posture or mobility parameter values 101. They may then also include structural and/or geometric parameter values of the connected sole. The new shoe parameter values 202 can in turn be used to design custom soles that will be perfectly adapted to the user when using a particular pair of shoes.

The shoe parameter values 201 202 can advantageously be stored in a database of the calculation computing device 30 or else on a third-party computing device 40. Indeed, it is provided that each connected sole 10 can be associated with a specific footwear item 11. By way of non-limiting example, the third-party computing device 40 can comprise a database indicating for each connected sole 10 with which model of footwear item said connected sole is associated. It is thus possible to associate shoe parameter values with a connected sole 10 and by extension to associate the data generated by said connected sole with a user equipped with a particular model of footwear item. As seen previously, the shoe parameter values 201 202 can include structural parameter values indicating the different elements constituting a model of predetermined footwear item 11. The shoe parameter values 201 can thus indicate the presence of a stiffener, a hard toe, a sole, a shank, a mounting insole, a sock liner, a stem. More particularly, the shoe parameter values 201 202 can describe the shape of each of the elements constituting a footwear item, their arrangement. Furthermore, they may also describe the mechanical characteristics for each element making up the footwear item, such as, by way of non-limiting examples, a thickness of the midsole, an abrasion resistance of the outer sole, a stiffness, insulation, or the damping properties of the midsole. The footwear item 201 202 parameter values may further include geometric parameter values. Said geometric parameter values can indicate the dimensions relating to each of the structural elements of the footwear item 11. Finally, the shoe parameter values 201 can also include aesthetic parameter values indicating for example, for each structural element a color, a type of material, a particular aesthetic pattern.

In order to provide a new sole adapted to the user and to the footwear item 11 for which said new sole is intended, a method 500 in accordance with the invention further comprises a step of calculating 550 one or more personalized parameter values of a new custom sole 301.

These personalized parameter values of a new custom sole 301 may include structural and geometric data on the sole. They may also advantageously include values relating to the characteristics and/or functionality of the sole such as potential damping, impact resistance, density, . . . .

This calculation step 550 may advantageously include the use of a learning model. Furthermore, such a learning model can be enhanced through reinforcement.

In particular, it may implement a learning model configured to refine the results according to the values that led to the selection of a personalized sole parameter value as well as comfort assessments formulated in return by the user(s). Learning can be a supervised or unsupervised learning. The method or system according to the invention is capable of implementing algorithms based on supervised or unsupervised learning methods. Thus, advantageously, the system or method for calculating personalized parameter values of the custom soles according to the invention is configured to train and implement one or more algorithms. These algorithms may have been built from different learning models, in particular supervised or unsupervised partitioning. The algorithm can be derived from the use of a supervised statistical learning model selected for example from kernel methods (e.g. Support Vector Machines SVM, Kernel Ridge Regression) described for example in Burges, 1998 (Data Mining and Knowledge Discovery. A Tutorial on Support Vector Machines for Pattern Recognition), ensemble methods (e.g. decision trees) described for example in Brieman, 2001 (Machine Learning. Random Forests), k-means partitioning, decision trees, logical regression or neural networks described for example in Rosenblatt, 1958 (The perceptron: a probabilistic model for information storage and organization in the brain) or else deep learning (Kernel Methods for Pattern Analysis Hardcover—Illustrated, Cambridge University Press, 2004; Machine learning techniques on ultra-low energy microcontrollers: TinyML, Machine Learning with TensorFlow Lite on Arduino and Ultra-Low-Power Microcontrollers, O'Reilly, 2020; Dimensionality reduction techniques for hyper-dimensional data, Topological Methods in Data Analysis and Visualization V: Theory, Algorithms, and Applications Mathematics and Visualization, Springer Veriag, 2020.) Alternatively, the invention may implement less complex models of the abacus type or decision rule of the expert system type.

Said personalized parameter values of the new sole can be calculated in particular from the posture or mobility parameter values 101 and the shoe parameter values 201. Indeed, one of the advantages of the invention is to allow to personalize the new sole according to the values of the posture or mobility parameters 101 while ensuring that said new sole can adapt to the footwear item 11 of the user. By way of non-limiting examples, the posture or mobility parameter values 101 can describe an impact force in contact with the ground, and a supination. The calculation step 550 can advantageously comprise a comparison of the value associated with the impact force, with the shoe parameter value describing an element of the footwear item having a cushioning function. Thus, as previously described, the shoe parameter values 201 may comprise a thickness of the midsole or more generally values describing damping properties adapted to a maximum impact force. In this case, the calculation step 550 then allows to determine that the posture or mobility parameter value 101 describing the impact force of the user is greater than the shoe parameter value 201 of the footwear item 11 describing a maximum impact force associated, for example, with the midsole of the footwear item 11. The calculation step 550 then allows to calculate a personalized parameter value of a new sole 301 in connection with the midsole. This personalized value can correspond to an increase in the thickness of the midsole present in the footwear item 11 for example. For a supinator user, such personalized values, related to the impact force, may only apply to the part of the midsole for which contact of the foot with the ground is detected, in particular on the outer part of the foot, that is to say in the area close to the little toe, namely the quintus.

As has been detailed, the step of calculating one or more personalized parameter values for a new custom sole may take the plantar morphology parameter values of the user and the activity parameter values of the user.

Furthermore, the step of calculating one or more personalized parameter values of a new custom sole may take into account other types of parameters such as robustness, safety or the environment (e.g. for aspects as damping and shock). For example, taking into account the environment (geolocation data or meteorological data) provided for the use of the custom sole will allow to adapt the personalized parameters of the sole so that, in addition to adapting to the user's gait, its plantar morphology, the anticipated activity with this sole and the characteristics of the footwear item intended to receive this sole, the environment of use of the sole is also taken into account.

In order to provide the user with a new personalized sole, a method 500 in accordance with the invention may include a step of generating 510 a value of a first comfort indicator Dx1 of a first sole, said first comfort indicator being generated following the entry of data by the user, via a man-machine interface of a presentation computing device 20. Indeed, it may be interesting to have data relating to the opinion of the user concerning the footwear item 11 adjusted with a connected sole 10 and worn by the user. In particular, provision is made for the user to be able, through a dedicated application, to select, on a presentation computing device 20, the model of footwear item corresponding to that equipped with the connected sole. By way of non-limiting example, the user can have access to the database of the third-party computing device 40 mentioned above. Once the footwear item 11 has been selected, the user can assign, for each shoe parameter value 201 of the footwear item 11, a rating, that is to say a numerical value, for example comprised between 0 and 5 or a decimal value as a fraction. From the values entered by the user, a value of a first comfort indicator is generated 510, such an indicator can in particular correspond to an average, weighted for each shoe parameter value 201 or not, calculated from the values entered by said user.

For the evaluation of comfort, it will be advantageous to use in particular scales called verbal scales (for example very low, low, average, . . . ) and not only numerical scales. These verbal scales can be on a numerical scale and can be used as a label in the context of learning.

During the step 550 of calculating one or more personalized parameter values of the new sole 301, said calculation step can then allow to take into account the generated value of the first comfort indicator. Preferably, the calculation step 550 can consist in calculating one or more personalized parameter values of the new sole 301 for the shoe parameter values whose value assigned by the user is less than a predetermined threshold and in keeping the shoe parameter values whose value assigned by the user is greater than a predetermined threshold. Thus, only the shoe parameter values 201 considered unsuitable for the user can be modified in order to provide the latter with a new highly personalized sole.

Once the value of the first comfort indicator Dx1 of a first sole has been generated 510, a method 500 in accordance with the invention may include a step of generating 515 a value of a second comfort indicator Dx2 of a second sole, said second comfort indicator being generated following the entry of data by the user of said second sole, via the man-machine interface of the presentation computing device 20. Indeed, it may be advantageous to have data relating to the user's opinion concerning a second footwear item, preferably worn by the user. In particular, provision is made for the user to be able, through a dedicated application, to select, on a presentation computing device 20, another model of footwear item. As mentioned previously in connection with the first comfort indicator Dx1, the user can have access to the database of the third-party computing device 40. Once the second footwear item has been selected, the user can assign, for each shoe parameter value of the second footwear item, a rating, that is to say a numerical value, for example comprised between 0 and 5 or else a decimal value in the form of a fraction. From the values entered by the user, a value of a second comfort indicator Dx2 is generated 515, such an indicator can in particular correspond to an average, weighted for each shoe parameter value or not, calculated from the values entered by said user.

During the step of calculating 550 one or more personalized parameter values of the new sole 301, said calculation step can then allow to take into account the generated value of the second comfort indicator. Preferably, the calculation step 550 can consist of calculating one or more personalized parameter values of the new sole 301 for the shoe parameter values 201 whose value assigned by the user in connection with the first comfort indicator Dx1 is below a predetermined threshold. The calculation step 550 can then allow to use the shoe parameter values of the second footwear item whose value assigned by the user is greater than a predetermined threshold to calculate the one or more personalized parameter values of the new sole 301.

Thus, the shoe parameter values of a second footwear item considered suitable for and by the user can be used to provide the latter with a new highly personalized sole.

One of the purposes of the invention is to provide a personalized sole directly adapted to the footwear item 11 of the user. For this purpose, a method 500 in accordance with the invention may comprise a step of receiving and analyzing 560 information from third-party users of third-party footwear items 11′, said third-party user information including for each third-party user of third-party footwear items:
values of third-party posture or mobility parameters 101′ calculated from raw data generated by at least one third-party connected sole 10′ associated with a third-party footwear item 11′, and
third-party shoe parameter values 201′ of the third-party footwear item 11′ associated with at least one third-party connected sole 10′.

Like the posture or mobility parameter values 101, shoe parameter values 201 associated with the connected sole 10, provision is made for one of the computing devices 20, 30, 40 to be able to store in memory a database referencing a plurality of information associated with third-party users. In an embodiment described in connection with FIG. 1, the calculation computing device 30 can directly access the database, through the communication network R1, hosted by the third-party computing device 40.

Once the third-party user information has been retrieved, a method 500 according to the invention may comprise a step of identifying 580 adjusted sole parameter values, said identification step including a comparison of the posture or mobility parameter values 101 generated by the connected sole 10 and shoe parameters 201 of the footwear item 11 associated with said connected sole to the previously analyzed third-party user information. Such a step 580 advantageously allows to identify a plurality of footwear items having third-party shoe parameter values 201′ substantially identical to the shoe parameter values 201 associated with the values of third-party posture or mobility parameters 101′. It is therefore possible to identify third-party shoe parameter values 201′ likely to be the most suitable for the personalized parameter values of the new sole 301. Indeed, it may be advantageous to modify the personalized parameter values of the new sole 301 in order to better adjust them to the footwear item 11 for which said new sole is intended. As seen previously, a personalized parameter value of a new sole 301 can correspond to an increase in the thickness of the midsole present in the footwear item 11 for example. However, the increase in the thickness of the midsole may be unsuitable for the footwear item 11 or at the very least generate discomfort for the user when using the footwear item 11 and cause injury during their use.

To include user experience data relating to each third-party footwear item 11′, said third-party user information may further include a satisfaction index value of a third-party user, preferably with respect to the third-party connected sole 10′ and/or to the footwear item 11′ including said third-party connected sole. Such a satisfaction index value may consist of a rating assigned by the third-party user, that is to say a numerical value, for example comprised between 0 and 5 or else a decimal value in the form of a fraction.

In this case, a method 500 in accordance with the invention can comprise a step of determining 570 a correlation index, for each third-party connected sole 10′, between the third-party posture or mobility parameter values 101′, the third-party shoe parameter values 201′ and the third-party user satisfaction index. For this purpose, such a determination step 570 may consist in comparing the third-party posture or mobility parameter values 101′ with reference posture or mobility parameter values. The reference posture or mobility parameter values correspond in particular to predetermined values for reference shoe parameter values in connection with a given footwear item. Thus, from the third-party shoe parameter values 201′, it is possible to determine reference posture or mobility parameter values, then to compare them with the third-party posture or mobility parameter values 101′. If the reference posture or mobility parameter values are substantially identical to the third posture or mobility parameter values 101′ then a correlation is established during the determination step 570 between the third-party posture or mobility parameter values 101′, the third-party shoe parameter values 201′ and the third-party user satisfaction index.

Alternatively, the step 570 of determining a correlation index may consist in comparing the use parameter value 401 calculated during the calculation step 535, as described previously, with a reference use parameter value associated with the footwear item 11. If the use parameter value 401 is substantially identical to the reference use parameter value then a correlation is established.

The establishment of a correlation can thus allow to take into account only the satisfaction indices of relevant third-party users, that is to say for which the use of the associated footwear item is in line with the use recommended by the manufacturer.

In order to provide a new sole 301 that is the most suitable for the footwear item 11 of the user, step 580 can advantageously lead to the identification of a change of material having damping properties adapted to the impact force of the user instead of the increase in thickness mentioned above.

In the case where such an identification takes place, a method 500 in accordance with the invention is advantageously configured to implement the step 550 of calculating one or more personalized parameter values of a new sole 301, said calculation step 550 further comprises a comparison of the posture or mobility parameter values 101 generated and the shoe parameter values 201 previously loaded with the user information previously analyzed. User information may, for example, correspond to physiognomic or physiological data, or to preferences or habits (e.g. practiced sports, level of activity, cushioning preference, . . . ).

In order to use only the most relevant third-party user information, that is to say that of third-party users having a use substantially identical to that of the user of the footwear item 11 equipped with the connected sole 10, the step 580 of identifying adjusted sole parameter values of a method 500 in accordance with the invention can take into account the at least one use parameter value calculated during step 535. In this case, when receiving and analyzing 560 information from third-party users of third-party footwear items 11′, the step 535 of calculating at least one use parameter value of a method 500 in accordance with the invention can advantageously be implemented from the values of third-party posture or mobility parameters 101′ calculated from raw data generated by at least one third-party connected sole 10′ associated with a third-party footwear item 11′. Like the use parameter value, a third-party use parameter value is determined in connection with each user information.

To stay as close as possible to the use made of a footwear item by the user, the step of identifying 580 adjusted sole parameter values can include the use of a repository specifying target values or target ranges for the personalized parameter values of the new sole 301 based on the posture or mobility parameter values 101 generated and the shoe parameter values 201 acquired. This advantageously allows to limit the personalized parameter values of the new sole 301 and to avoid, by the use of said repository, selecting aberrant or at least unsuitable personalized values. For this purpose, such a repository can comprise reference posture or mobility parameter values and reference shoe parameter values and minimum and maximum reference deviations for each of said values. Indeed, and in particular in the context of the comparison with the information of third-party users, the latter may comprise one or more values of third-party posture or mobility parameters 101′ and one or more third-party shoe parameter values 201′ of the third-party footwear item that may or may not be considered usable. The use of such a reference allows to define values or ranges of values for which a personalized parameter value of the new sole 301 can be used. Thus, a calculation step 550 of a method 500 in accordance with the invention could advantageously allow to optimize one or more personalized parameter values of a new sole 301, on the basis of a deviation between the posture or mobility parameter values 101 generated and the reference posture or mobility parameter values and/or on the basis of a deviation between the personalized shoe parameter values 201 acquired and the personalized parameter values of a new sole 301. For example, if a posture or mobility parameter value 101 or a personalized parameter value of the new sole 301, is not substantially identical to a reference posture or mobility parameter value or to a reference shoe parameter value, but said value is comprised within a corresponding minimum or maximum reference deviation, then the personalized parameter value of the new sole 301 is maintained. In the event that said value is not comprised within a corresponding minimum or maximum reference deviation, it is the corresponding reference value which is used instead of the personalized parameter value of the new sole 301 calculated.

In order to take into consideration only the most relevant third-party user information, the step 580 of identifying adjusted sole parameter values of a method according to the invention can be implemented only for the information from third-party users including a satisfaction index value and/or a correlation index respectively greater than a predetermined threshold. For this purpose, the identification step 580 can advantageously comprise an operation of filtering information from third-party users according to a predetermined threshold satisfaction index value. The third-party user information thus filtered can then be the subject of a second filtering operation, during the identification step 580, depending on the correlation index.

In order to facilitate the manufacture of a new sole, in particular by 3D printing techniques, a method according to the invention includes a step of generating, from one or more personalized parameter values of the new sole 301, a digital model of the new sole. The digital model according to the present invention can be a three-dimensional model of the new sole comprising a functional description. The functional description can include information such as the materials used, where said materials are placed at the new sole.

The digital model can be a three-dimensional model of a footwear item including the new sole, such as for example a digital model in a Computer Aided Design system. The digital model can comprise information on the materials used, their shape and their dimensions.

The digital model of the new sole can also be personalized according to data entered by the user. For example, the user may be asked which sensor(s) the new personalized sole should comprise.

As mentioned earlier, raw data are generally generated for a given period of time and according to the gait of a user. Gait analysis could extend over the lifespan of the shoe or user. In this case, long-term trends, deviations and changes in the gait cycle of the user during said cycle can be determined.

Thus, the connected sole 10 can also be used in the context of monitoring the user's gait, on a daily basis. To this end, the connected sole 10 can transmit the raw data from the sensors to a computing device such as one of those already mentioned. Once the raw data has been processed, the posture or mobility parameter values 101 can then be presented to the user as a record of their daily activities on a presentation computing device 20. For example, the number of paces taken during a day could be extracted from the sensor data and presented to the person. Furthermore, it is possible to calculate the energy expenditure from the appropriate raw data and present the daily energy expenditure to the person. Other information that may be presented to the person as part of a daily activity tracker includes the walking and/or running distance, the walking and/or running time, fastest speed of the day, etc.

As part of a daily activity monitoring service, it is also possible to implement weight monitoring on the basis of the raw data from suitable sensors of the connected sole 10. For example, the raw data from the sensor could include pressure information from a pressure sensor that could be used to determine the user's weight. It is then possible to present the daily weight to the user.

Based on the determined weight, a specific midsole and/or outer sole with specific properties or materials could be modified or chosen. For example, the thickness of the midsole could be adapted to the weight of the user and the heavier the person, the thicker the midsole could be to provide sufficient cushioning. The outer sole could be made more abrasion resistant if the person is rather heavy to counter wear over time.

According to another aspect, the invention relates to a method 600 for manufacturing a new custom sole by a three-dimensional printing device, said method including a step of downloading 610, by the printing device, a configuration file X1 including one or more personalized parameter values of the new sole 301 calculated according to a method 500 for calculating personalized parameter values of a new sole according to the invention. Such a configuration file is transmitted by the calculation computing device 30 and can be in the form of an STL (for STereo-lithography) format file. Provision is also made for the method 500 for calculating personalized parameter values of a new sole to include a step of transmitting the configuration file X1 directly to the three-dimensional printing device and, upon reception of said configuration file X1 by the three-dimensional printing device, for the method 600 for manufacturing a new sole to be implemented.

A method 600 according to the invention further comprises a step of generating 620, from the personalized parameter values of the new sole 301, a digital model of the new sole. Thus, the new sole can be presented to the user or the manufacturer. For example, a 3D view of the model of the new sole can be presented on a display screen, by way of non-limiting example in a window of a web browser or any other appropriate program. It is also possible to present such a digital model via a dedicated application accessible from a presentation computing device 20.

Once the digital model has been generated, a method 600 in accordance with the invention comprises a step of printing 630 a new sole from the generated digital model. The printing step 630 is advantageously carried out by selective laser sintering or else by photopolymerization of photosensitive resins with UV rays, or more generally via any suitable three-dimensional printer.

The sole thus manufactured may or may not be a connected sole. An electronic unit such as units 1001 or 1002 can then be provided to equip the custom sole.

1. A method for calculating personalized parameter values of custom soles for a user, said calculation method including execution, by one or more computing devices (20, 30, 40), of the following steps:
obtaining posture or mobility parameter values of the user, said posture or mobility parameter values having been calculated from raw data generated by at least one connected sole associated with a footwear item used by said user;
obtaining plantar morphology parameter values of the user;
obtaining; user activity parameter values;
obtaining new shoe parameter values, said new shoe parameter values including structural and/or geometric parameter values of a footwear item intended to receive the custom soles; and
a step of calculating one or more personalized parameter values of the custom soles for the user, said personalized parameter value(s) being calculated from the posture or mobility parameter values, the plantar morphology parameter values of the user, user activity parameter values and structural and/or geometric parameter values of the footwear item intended to receive the custom soles.

2. The calculation method according to the claim 1, further comprising loading old shoe parameter values, said old shoe parameter values including structural and/or geometric parameter values of the footwear item used by the user, associated with the connected sole, wherein said personalized parameter value(s) of the custom soles are further calculated from the old shoe parameter values.

3. The calculation method according to one of claim 1, comprising:
receiving and analyzing information from third-party users of third-party footwear items, said third-party user information including for each third-party user of third-party footwear items:
third-party posture or mobility parameter values calculated from raw data generated by at least one third-party connected sole associated with a third-party footwear item, and
old third-party shoe parameter values of the third-party footwear item associated with said at least one third-party connected sole;
identifying adjusted sole parameter values, said identification step including a comparison of the posture or mobility parameter values of the user, with information from said third-party users analyzed beforehand.

4. The calculation method according to claim 3, wherein said third-party user information further includes a satisfaction index value of a said third-party user, with respect to the third-party connected sole and/or the third-party footwear item including said third-party connected sole.

5. The calculation method according to claim 4, further comprising determining a correlation index, for each third-party connected sole, between the third-party posture or mobility parameter values, the third-party shoe parameter values, and the third-party user satisfaction index.

6. The calculation method according to claim 4, wherein said identifying adjusted sole parameter values is implemented only for third-party user information including a satisfaction index value and/or a correlation index respectively greater than a predetermined threshold.

7. The calculation method according claim 3, wherein said identifying adjusted sole parameter values includes use of repository specifying target values or target ranges for the personalized parameter values of the custom sole based on the posture or mobility parameter values and the shoe parameter values.

8. The calculation method according to claim 1, further comprising, prior to obtaining posture or mobility parameter values of the user, processing raw data generated by the at least one connected sole used by the user, said processing allowing generation of said posture or mobility parameter values.

9. The calculation method according to claim 1, wherein the posture or mobility parameter values are selected from impact force in contact with the ground, pronation, supination and/or lameness parameters.

10. The calculation method according to claim 1, further comprising generating a value of a first comfort indicator of a first sole, said first comfort indicator being generated following entry of data by the user, via a man-machine interface of a presentation computing device, wherein said calculating one or more personalized parameter values of the custom sole takes into account the generated value of the first comfort indicator.

11. The calculation method according to claim 10, further comprising generating a value of a second comfort indicator of a second sole, said second comfort indicator being generated following entry of data by the user of said second sole, via the man-machine interface of the presentation computing device, wherein said calculating one or more personalized parameter values of the custom sole, takes into account the generated value of the second comfort indicator.

12. The calculation method according to claim 1, wherein the posture or mobility parameter values have been calculated by one or more processors integrated into the at least one connected sole.

13. A method for manufacturing a custom sole by a three-dimensional printing device, comprising:
Loading, by the printing device, a configuration file including one or more personalized parameter values of the custom sole calculated according to the calculation method of claim 1;
Generating, from the personalized parameter values of the custom sole, a digital model of the custom sole; and
Printing a custom sole from the generated digital model.

14. A system for calculating personalized parameter values of a custom sole for the design of custom soles, said system comprising at least one calculation computing device, said at least one calculation computing device being configured to: Obtain posture or mobility parameter values of a user, said posture or mobility parameter values having been calculated from raw data generated by at least one connected sole associated with a footwear item used by said user; Obtain plantar morphology parameter values of the user; Obtain user activity parameter values; Obtain new shoe parameter values, said new shoe parameter values including structural and/or geometric parameter values of a footwear item intended to receive the custom soles; and Calculate one or more personalized parameter values of the custom soles for the user, said personalized parameter value(s) being calculated from the posture or mobility parameter values, the plantar morphology parameter values of the user, user activity parameter values and structural and/or geometric parameter values of the footwear item intended to receive the custom soles.

15. A three-dimensional printing system for the manufacture of new custom soles, said system including:
a processor configured to:
obtain posture or mobility parameter values, plantar morphology parameter values of a user, user activity parameter values and structural and/or geometric parameter values of a footwear item intended to receive the custom soles,
calculate one or more personalized parameter values of the custom soles for the user from the parameter values obtained,
generate a digital model of the custom sole; and
a printing device configured to print the custom soles from the generated digital model.

16. The three-dimensional printing system according to claim 15, further comprising a scanner capable of generating parameter values of the plantar morphology of the user.
